(19) United States
(12) Patent Application Publication
(10) Pub. No.: US 2025/0144252 A1
(43) Pub. Date: May 8, 2025

(54) CARBON NANOPARTICLE SUSPENSION INJECTION-FE, PREPARATION METHOD, APPLICATION AND USE METHOD

(71) Applicant: Sichuan Enray Pharmaceutical Technology Co., LTD., Chengdu (CN)

(72) Inventors: Xiaohai Tang, Chengdu (CN); Kexin Tang, Chengdu (CN); Yuanfang Huang, Chengdu (CN); Cheng Zeng, Chengdu (CN)

(21) Appl. No.: 18/970,315

(22) Filed: Dec. 5, 2024

Related U.S. Application Data
(63) Continuation of application No. PCT/CN2024/082864, filed on Mar. 21, 2024.

(30) Foreign Application Priority Data
Mar. 31, 2023 (CN) 2023103382721
Mar. 29, 2024 (CN) 2024103716958

(51) Int. Cl. A61K 49/18; A61K 9/00; A61K 47/12; A61K 47/34; A61K 49/08

(52) U.S. Cl. CPC A61K 49/1818; A61K 9/0019; A61K 47/12; A61K 47/34; A61K 49/08

(57) ABSTRACT

Carbon nanoparticle suspension injection-Fe, a preparation method, an application and a use method. The carbon nanoparticle suspension injection-Fe includes carbon nanoparticle suspension injection and ferrous sulfate, where a concentration of the carbon nanoparticle suspension injection is 20-100 mg/mL, a concentration of ferrous ions in the carbon nanoparticle suspension injection-Fe is 0.5-60 mg/mL, and a particle size of carbon nanoparticles in the carbon nanoparticle suspension injection-Fe is 90-250 nm, and a pH value is 2.8-6.0. The carbon nanoparticle suspension injection-Fe serves as a developing agent after being injected into a tumor to show distribution of the carbon nanoparticle suspension injection-Fe and retention in the tumor on a magnetic resonance image.

### CROSS-REFERENCE TO RELATED APPLICATIONS

This application is a continuation application of PCT/CN2024/082864. This application claims priorities from PCT Application No.: PCT/CN2024/082864, filed on Mar. 21, 2024, Chinese patent application No. 202310338272.1, filed on Mar. 31, 2023, and from 2024103716958, filed on Mar. 29, 2024, the entire content of which are incorporated herein by reference.

### TECHNICAL FIELD

The present disclosure belongs to the field of medicine, and particularly relates to carbon nanoparticle suspension injection-Fe, a preparation method, an application and a use method.

### BACKGROUND

Carbon nanoparticle suspension injection-Fe (CNSI-Fe) is an innovative anticancer drug with carbon nanoparticles as a carrier and divalent iron ions or trivalent iron ions as an effective component. The mechanism of action is that after CNSI-Fe is locally injected into cancer tissue, and enters cancer cells through excessive iron channels expressed on cancer cell membranes. A large amount of iron ions enter the cancer cells rich in hydrogen peroxide (H₂O₂) and then a Fenton reaction occurs with H₂O₂ to produce a large number of hydroxyl free radicals (·OH), ·OH has an extremely strong oxidation property, and reacts with unsaturated polyfatty acids (UPFAs) in the cells to produce a large number of extremely destructive lipid hydrogen peroxide (L-OOH), namely lipid-reactive oxygen species (Lipid-ROS), and Lipid-ROS can destroy organelles and lead to cell destruction, triggering Ferroptosis.

CNSI-Fe²⁺+H₂O₂→ROS(·OH)→L-ROS→Ferroptosis

In preliminary preclinical animal trials, the carbon nanoparticle suspension injection-Fe shows excellent anti-cancer efficacy, and has a good therapeutic effect on colorectal cancer, lung cancer, breast cancer, pancreatic cancer, undifferentiated thyroid cancer and other solid cancers. Moreover, the carbon nanoparticle suspension injection-Fe has the characteristics of good tolerance, high safety and overcoming multidrug resistance. As a Class 2 anticancer drug, the carbon nanoparticle suspension injection-Fe has been granted invention patents in China, the United States and Japan (patent name: a carbon nanoparticle suspension injection-Fe composite system and composite thereof, preparation method and application), which has four main functions such as tumor localization, lymphatic tracing, treatment of primary cancer lesions and metastatic lymph nodes, and has synergistic effects with various existing chemotherapy drugs, radiotherapy, hyperthermia and other treatments.

Intratumoral administration of the carbon nanoparticle suspension injection-Fe: Fe²⁺ is targeted in tumor tissue by using the carrier effect of the carbon nanoparticle suspension injection-Fe, and the effect of tumor reduction is achieved through the mechanism of “Ferroptosis”. The preoperative positioning function of the carbon nanoparticle suspension injection-Fe is conductive to accurate positioning or follow-up observation of surgery. The lymphatic tracing effect of the carbon nanoparticle suspension injection-Fe is utilized to stain tumor lymphatic tissue black, and accurate resection can be performed in subsequent surgery.

Existing tumor diagnosis techniques, such as B-ultrasound, computerized tomography (CT), and magnetic resonance imaging (MRI), can observe the location and size of a tumor, but whether the drug enters the tumor cannot be displayed. There are also some new compounds that can be used for the diagnosis and treatment of tumors, such as novel nanoscale photosensitizers that self-assemble silicon phthalocyanine (PcM) with albumin, which can achieve tumor-targeted fluorescence imaging and photodynamic immunotherapy; and nanoparticles made of iron trioxide nanocrystals combined with folic acid and doxorubicin, which can be used for magnetic resonance imaging and cancer therapy.

The carbon nanoparticle suspension injection-Fe in the prior art has a good curative effect when being applied to anti-tumor treatment, but the administration mode is intratumoral injection, and an optimal curative effect can be achieved only when the drug is uniformly distributed during injection administration. After intratumoral administration of the carbon nanoparticle suspension injection-Fe, only distribution of the carbon nanoparticle suspension injection-Fe on the tumor surface can be observed, and distribution of the carbon nanoparticle suspension injection-Fe in the tumor cannot be observed, which may cause uneven distribution of the carbon nanoparticle suspension injection-Fe and affect efficacy of the carbon nanoparticle suspension injection-Fe. Moreover, the tumor is located in a human body, and the amount and retention of the carbon nanoparticle suspension injection-Fe in a tumor site cannot be known.

### SUMMARY

An objective of the present disclosure is to provide an application of carbon nanoparticle suspension injection-Fe, so as to solve the problems that in the prior art, after intratumoral administration of the carbon nanoparticle suspension injection-Fe, only distribution of the drug on the tumor surface can be observed, and distribution of the carbon nanoparticle suspension injection-Fe in the tumor cannot be observed, which may cause uneven distribution of the carbon nanoparticle suspension injection-Fe and affect efficacy of the carbon nanoparticle suspension injection-Fe; and the tumor is located in a human body, and the amount and retention of the carbon nanoparticle suspension injection-Fe in a tumor site cannot be known.

In order to solve the above technical problems, based on some examples, the present disclosure provides an application of carbon nanoparticle suspension injection-Fe. The carbon nanoparticle suspension injection-Fe is prepared by mixing carbon nanoparticle suspension injection with ferrous sulfate for injection. A concentration of the carbon nanoparticle suspension injection is 20-100 mg/mL, and a concentration of ferrous ions in the carbon nanoparticle suspension injection-Fe is 0.5-60 mg/mL.

The carbon nanoparticle suspension injection-Fe serves as a developing agent after being injected into a tumor to show distribution of the carbon nanoparticle suspension injection-Fe in the tumor during magnetic resonance imaging.

Further, the concentration of the carbon nanoparticle suspension injection is 50 mg/mL, and the concentration of the ferrous ions is 15 mg/mL.

Further, a defoaming agent is contained in the carbon nanoparticle suspension injection.

Further, a particle size of carbon nanoparticles in the carbon nanoparticle suspension injection is 90-250 nm, and a pH value is 2.8-6.0.

Further, the defoaming agent is dimethicone.

Each 1000 mL of the carbon nanoparticle suspension injection includes:

20-100 g of carbon nanoparticles, 17-30 g of poloxamer, 2-4 g of sodium citrate, 8-10 g of sodium chloride, 0.05-0.5 g of dimethicone, and the balance water for injection.

Further, each 1000 mL of the carbon nanoparticle suspension injection includes: 50 g of carbon nanoparticles, 20 g of poloxamer, 0.2 g of dimethicone, 3 g of sodium citrate, 9 g of sodium chloride, and the balance water for injection.

Further, the carbon nanoparticles are carbon black C40.

Further, the preparation process for the carbon nanoparticles includes:
degreasing carbon nanoparticles with ethyl acetate; washing the carbon nanoparticles with nitric acid, and then, washing the carbon nanoparticles with water until a pH value of the carbon nanoparticles is stable; and washing the carbon nanoparticles with sodium hydroxide, and then washing same with water until the pH value of the carbon nanoparticles is stable.

Further, the preparation process for each part of the carbon nanoparticle suspension injection includes:
weighing 60 mg of sodium citrate, 400 mg of poloxamer and 4 mg of dimethicone, dissolving same in 20 mL of normal saline, then, adding 1000 mg of carbon nanoparticles, performing homogenization with a homogenizer for 5-10 minutes, and transferring the product to a fine homogenizer for fine homogenization multiple times after completion of homogenization.

Further, the preparation process for each part of ferrous sulfate includes: dissolving 1490 mg of ferrous sulfate heptahydrate in 20 ml of water for injection, and adjusting a pH value of the solution with sulfuric acid to 2.8 after completion of dissolution, performing packaging, performing freeze-drying, backwashing nitrogen, and pressing lips, thereby obtaining the ferrous sulfate.

The above-mentioned technical solution of the present disclosure at least has the following beneficial technical effects:

The carbon nanoparticle suspension injection-Fe is used in combination with magnetic resonance imaging, which can guide intratumoral administration of the carbon nanoparticle suspension injection-Fe, such that the carbon nanoparticle suspension injection-Fe is uniformly distributed in the tumor after administration, and an anticancer effect of the carbon nanoparticle suspension injection-Fe is improved. Moreover, retention of the carbon nanoparticle suspension injection-Fe at different time after administration can also be observed through magnetic resonance imaging (MRI), so as to know the amount and retention time of the carbon nanoparticle suspension injection-Fe in the tumor, and the retention time of the carbon nanoparticle suspension injection-Fe is long, which can effectively cover multiple mitotic cycles of cancer cells.

### BRIEF DESCRIPTION OF THE DRAWINGS

In order to more clearly illustrate technical solutions in the examples of the present disclosure or in the prior art, a brief introduction to the accompanying drawings required for the description of the examples will be provided below. Obviously, the accompanying drawings in the following description are only some of the examples of the present disclosure, and those of ordinary skill in the art would also be able to derive other drawings from these drawings without making creative efforts.

FIG. 1 shows a magnetic resonance imaging (MRI) image of carbon nanoparticle suspension injection-Fe of different concentrations in an example of the present disclosure.

FIG. 2 shows an MRI image before intratumoral administration in mice in an example of the present disclosure.

FIG. 3 shows an MRI image on day 0 after intratumoral administration in mice of carbon nanoparticle suspension injection-Fe in an example of the present disclosure.

FIG. 4 shows an MRI image on day 3 after intratumoral administration in mice of carbon nanoparticle suspension injection-Fe in an example of the present disclosure.

FIG. 5 shows an MRI image on day 14 after intratumoral administration in mice of carbon nanoparticle suspension injection-Fe in an example of the present disclosure.

### DETAILED DESCRIPTIONS OF THE EMBODIMENTS

At present, the following problems exist in the prior art: after intratumoral administration of the carbon nanoparticle suspension injection-Fe, only distribution of the carbon nanoparticle suspension injection-Fe on the tumor surface can be observed, and distribution of the carbon nanoparticle suspension injection-Fe in the tumor cannot be observed, which may cause uneven distribution of the carbon nanoparticle suspension injection-Fe and affect efficacy of the carbon nanoparticle suspension injection-Fe; and the tumor is located in a human body, and the amount and retention of the carbon nanoparticle suspension injection-Fe in a tumor site cannot be known.

In order to solve the above technical problems, an example of the present disclosure provides an application of carbon nanoparticle suspension injection-Fe. The carbon nanoparticle suspension injection-Fe is prepared by mixing carbon nanoparticle suspension injection with ferrous sulfate. A concentration of the carbon nanoparticle suspension injection is 20-100 mg/mL, and a concentration of ferrous ions in the carbon nanoparticle suspension injection-Fe is 0.5-60 mg/mL. Optionally, measurement is performed on a concentration of the ferrous sulfate in the carbon nanoparticle suspension injection-Fe, and a concentration range of the ferrous sulfate may be 0.5-60 mg/mL.

Preferably, the concentration of the carbon nanoparticle suspension injection is 50 mg/mL, and the concentration of the ferrous ions is 15 mg/mL.

The carbon nanoparticle suspension injection-Fe serves as a developing agent after being injected into a tumor to show distribution of the carbon nanoparticle suspension injection-Fe in the tumor during magnetic resonance imaging.

In this example, the carbon nanoparticle suspension injection-Fe (CNSI-Fe) is composed of two parts, composition 1 is ferrous sulfate for injection, and composition 2 is special solvent carbon nanoparticle suspension injection. When in use, the ferrous sulfate for injection is dissolved in the special solvent carbon nanoparticle suspension injection to obtain the carbon nanoparticle suspension injection-Fe. The carbon nanoparticle suspension injection-Fe is a kind of nanometer suspension anticancer medicine, which takes carbon nanoparticles as a carrier and divalent iron ions or trivalent iron ions as an effective component. Since the carbon nanoparticle suspension injection-Fe contains iron ions, the carbon nanoparticle suspension injection-Fe can be developed in a magnetic resonance imaging system. The carbon nanoparticle suspension injection-Fe is used in combination with magnetic resonance imaging, which can guide administration of the carbon nanoparticle suspension injection-Fe, such that the carbon nanoparticle suspension injection-Fe is evenly distributed in a tumor, thus achieving the function of enhancing an anticancer effect of the carbon nanoparticle suspension injection-Fe. Moreover, the amount and retention time of the carbon nanoparticle suspension injection-Fe in the tumor can also be observed.

Specific operations are: Before the first administration of the carbon nanoparticle suspension injection-Fe, a location and a size of the tumor are determined through magnetic resonance imaging (MRI), so as to facilitate the administration of the carbon nanoparticle suspension injection-Fe. Before the next administration, the size of the tumor and distribution of the carbon nanoparticle suspension injection-Fe in the tumor are determined through MRI, and the administration of the carbon nanoparticle suspension injection-Fe is guided according to the distribution of the carbon nanoparticle suspension injection-Fe in the tumor, such that the carbon nanoparticle suspension injection-Fe can be injected into the tumor site without carbon nanoparticle suspension injection-Fe distribution, so as to achieve the purpose of uniform distribution of the carbon nanoparticle suspension injection-Fe in the tumor and give full play to the anticancer effect. Later, MRI is employed to determine the size of the tumor and the distribution of the carbon nanoparticle suspension injection-Fe in tumor before each administration to ensure the uniform distribution of the carbon nanoparticle suspension injection-Fe in the tumor. Moreover, retention of the carbon nanoparticle suspension injection-Fe at different time after administration can also be observed through MRI, so as to know the amount and retention time of the carbon nanoparticle suspension injection-Fe in the tumor.

The carbon nanoparticle suspension injection-Fe is used in combination with magnetic resonance imaging, which can guide the intratumoral administration of the carbon nanoparticle suspension injection-Fe, such that the carbon nanoparticle suspension injection-Fe is uniformly distributed in the tumor after administration, and the anticancer effect of the carbon nanoparticle suspension injection-Fe is improved. Moreover, the retention of the carbon nanoparticle suspension injection-Fe at different time after administration can also be observed through MRI, so as to know the amount and retention time of the carbon nanoparticle suspension injection-Fe in the tumor, and the retention time of the carbon nanoparticle suspension injection-Fe is long, which can effectively cover multiple mitotic cycles of cancer cells.

In an example of the present disclosure, the carbon nanoparticle suspension injection contains a defoaming agent used for eliminating influence of bubbles generated during suction of the carbon nanoparticle suspension injection and facilitating practical operations.

Further, a particle size of carbon nanoparticles in the carbon nanoparticle suspension injection is 90-250 nm, and a pH value is 2.8-6.0.

Further, the defoaming agent is dimethicone. Each 1000 mL of the carbon nanoparticle suspension injection includes:
20-100 g of carbon nanoparticles, 17-30 g of poloxamer, 0.05-0.5 g of dimethicone, 2-4 g of sodium citrate, 8-10 g of sodium chloride, and the balance water for injection. Preferably, each 1000 mL of the carbon nanoparticle suspension injection includes: 50 g of carbon nanoparticles, 20 g of poloxamer, 0.2 g of dimethicone, 3 g of sodium citrate, 9 g of sodium chloride, and the balance water for injection.

Further, the carbon nanoparticles are carbon black C40.

In an example of the present disclosure, the preparation process for the carbon nanoparticles includes:
degreasing carbon nanoparticles with ethyl acetate; washing the carbon nanoparticles with nitric acid, and then, washing the carbon nanoparticles with water until a pH value of the carbon nanoparticles is stable; and washing the carbon nanoparticles with sodium hydroxide, and then washing same with water until the pH value of the carbon nanoparticles is stable.

In an example of the present disclosure, the preparation process for each 20 mL of the carbon nanoparticle suspension injection includes:
400-2000 mg of carbon nanoparticles, 340-600 mg of poloxamer, 1-10 mg of dimethicone, 40-80 mg of sodium citrate, 160-200 mg of sodium chloride, and the balance water for injection. Preferably, 60 mg of sodium citrate, 400 mg of poloxamer and 4 mg of dimethicone are weighed and dissolved in 20 mL of normal saline. Then, 1000 mg of carbon nanoparticles are added. Homogenization is performed with a homogenizer for 5-10 minutes at a rotation speed of 7000 rpm. The product is transferred to a fine homogenizer for fine homogenization multiple times after completion of homogenization. Preferably, fine homogenization is performed three times at a homogenization pressure of 20000 psi.

Further, the preparation process for each part of ferrous sulfate includes: 1490 mg of ferrous sulfate heptahydrate is dissolved in 20 ml of water for injection, and a pH value of the solution is adjusted with sulfuric acid to 2.8 after completion of dissolution. Packaging and freeze-drying are performed. Nitrogen is backwashed, and lips are pressed, thereby obtaining the ferrous sulfate. Preferably, 91 mg of ferrous sulfate is in each bottle.

Finally, the carbon nanoparticle suspension injection is sucked and added into the ferrous sulfate for injection, and uniformly mixed to obtain the carbon nanoparticle suspension injection-Fe.

It should be noted that before the use of the carbon nanoparticle suspension injection-Fe, the carbon nanoparticle suspension injection and the ferrous sulfate are separately packaged, and on-site configuration is carried out at the time of use. Specific configuration parameters are as follows:

When the concentration of the ferrous ions is 3.75 mg/mL, 7.5 mg/mL, 15 mg/mL, 30 mg/mL and 60 mg/mL respectively, 8 mL, 4 mL, 2 mL, 1 mL and 0.5 mL of the carbon nanoparticle suspension injection are taken with syringes respectively. The syringes pass through rubber stoppers of ferrous sulfate bottles for injection under the condition of isolating air, and the carbon nanoparticle suspension injection is injected into the ferrous sulfate bottle to be mixed and shaken well. After mixing for 5 minutes, the mixture is shaken well to obtain the carbon nanoparticle suspension injection-Fe. Ferrous sulfate in each bottle is 91 mg, a ferrous dose is 30 mg, and specific corresponding parameter relationship is as follows:

In the case that the concentration of the ferrous ions is 60 mg/mL, 0.5 mL of carbon nanoparticle suspension injection is taken with a syringe, and the syringe passes through a rubber stopper of a ferrous sulfate bottle for injection under the condition of isolating air. The carbon nanoparticle suspension injection is injected into the ferrous sulfate bottle to be mixed, and shaken well. After mixing for 5 minutes, the mixture is shaken well to obtain the carbon nanoparticle suspension injection-Fe.

In the case that the concentration of the ferrous ions is 30 mg/mL, 1 mL of carbon nanoparticle suspension injection is taken with a syringe, and the syringe passes through a rubber stopper of a ferrous sulfate bottle for injection under the condition of isolating air. The carbon nanoparticle suspension injection is injected into the ferrous sulfate bottle to be mixed, and shaken well. After mixing for 5 minutes, the mixture is shaken well to obtain the carbon nanoparticle suspension injection-Fe.

In the case that the concentration of the ferrous ions is 15 mg/mL, 2 mL of carbon nanoparticle suspension injection is taken with a syringe, and the syringe passes through a rubber stopper of a ferrous sulfate bottle for injection under the condition of isolating air. The carbon nanoparticle suspension injection is injected into the ferrous sulfate bottle to be mixed, and shaken well. After mixing for 5 minutes, the mixture is shaken well to obtain the carbon nanoparticle suspension injection-Fe.

In the case that the concentration of the ferrous ions is 7.5 mg/mL, 4 mL of carbon nanoparticle suspension injection is taken with a syringe, and the syringe passes through a rubber stopper of a ferrous sulfate bottle for injection under the condition of isolating air. The carbon nanoparticle suspension injection is injected into the ferrous sulfate bottle to be mixed, and shaken well. After mixing for 5 minutes, the mixture is shaken well to obtain the carbon nanoparticle suspension injection-Fe.

In the case that the concentration of the ferrous ions is 3.75 mg/mL, 8 mL of carbon nanoparticle suspension injection is taken with a syringe, and the syringe passes through a rubber stopper of a ferrous sulfate bottle for injection under the condition of isolating air. The carbon nanoparticle suspension injection is injected into the ferrous sulfate bottle to be mixed, and shaken well. After mixing for 5 minutes, the mixture is shaken well to obtain the carbon nanoparticle suspension injection-Fe.

The following tests are conducted for the carbon nanoparticle suspension injection-Fe in combination with MRI and an analysis process in the present disclosure:

(1) In vitro MRI of carbon nanoparticle suspension injection-Fe

Test materials: carbon nanoparticle suspension injection, and ferrous sulfate for injection

Test method: a series of carbon nanoparticle suspension injection with different concentrations of ferrous ions were prepared, scanning was performed on a 7T magnetic resonance scanning system for small animals, and imaging colors and T2 values of the carbon nanoparticle suspension injection-Fe with different iron concentrations were observed.

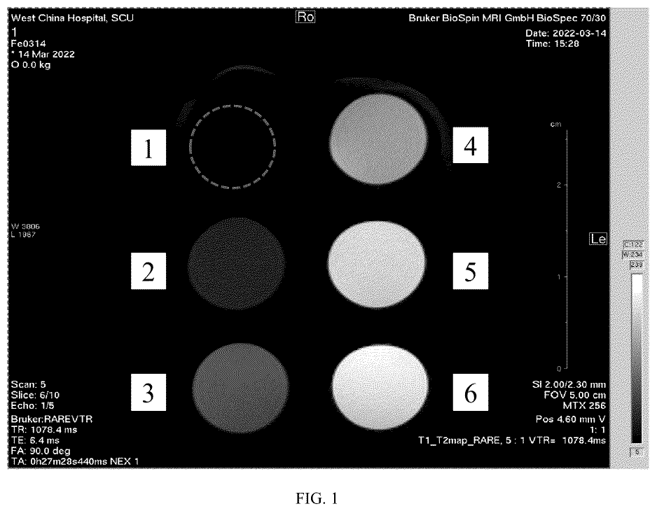

Test results: the carbon nanoparticle suspension injection-Fe can be imaged in the MRI system, imaging is mainly related to iron ions, and the higher the iron concentration, the darker the imaging color and the smaller the T2 value. Specific results are shown in Table 1 and FIG. 1.

TABLE 1
Ferrous ion concentration and T2 value of
carbon nanoparticle suspension injection-Fe

| Serial number | 1 | 2 | 3 | 4 | 5 | 6 |
|---|---|---|---|---|---|---|
| Ferrous ion concentration | 15 | 7.5 | 3.75 | 1.875 | 0.9375 | 0.4687 |
| (mg/mL) | | | | | | |
| T2 mean value (ms) | — | 6.63 | 8.05 | 13.8 | 27.9 | 87.2 |
| SD (ms) | — | 1.96 | 0.814 | 0.526 | 0.89 | 3.89 |

FIG. 1 also shows that the carbon nanoparticle suspension injection-Fe can be imaged in the magnetic resonance system, and the higher the iron concentration, the darker the imaging color. In FIG. 1, serial number of 1 represents that the ferrous ion concentration is 15 mg/mL, and the color is black. Serial number of 2 represents that the ferrous ion concentration is 7.5 mg/mL, and the color is dark gray. Serial number 3 represents that the ferrous ion concentration is 3.75 mg/mL, and the color is gray. The color is lighter and lighter with a decrease of the ferrous ion concentration.

(2) In vivo MRI of carbon nanoparticle suspension injection-Fe

Test materials: murine colon cancer CT26.WT cells, RMPI1640 medium for cells, fetal bovine serum (FBS), cell digestion solution trypsin, penicillin streptomycin mixture, phosphate buffer solution (PBS, pH 7.4), and carbon nanoparticle suspension injection-Fe (carbon nanoparticle-ferrous sulfate, carbon nanoparticles: ferrous ions=50:15 mg/ml).

Test animals: SPF Balb/c mice, female, 4-6 weeks old, weighing 20±2 g. Free water drinking and eating were carried out during the test. The mice were kept in isolation cages with independent air supply and exposed to light for 12 hours a day, and 5 mice were in one cage.

Test methods: CT26.WT colon cancer cells in a logarithmic growth phase were collected, the concentration of cell suspension was adjusted to 3×10⁷ cells/mL, and the cells were inoculated subcutaneously in the right upper limb of Balb/c mice at 0.1 mL/mouse (containing about 3×10⁶ cells). When a diameter of a tumor of the inoculated mouse was 7-8 mm, the carbon nanoparticle suspension injection-Fe was injected into the tumor, and on day 0, 3 and 14 after administration, scanning was performed with a 7T magnetic resonance scanning system for small animals.

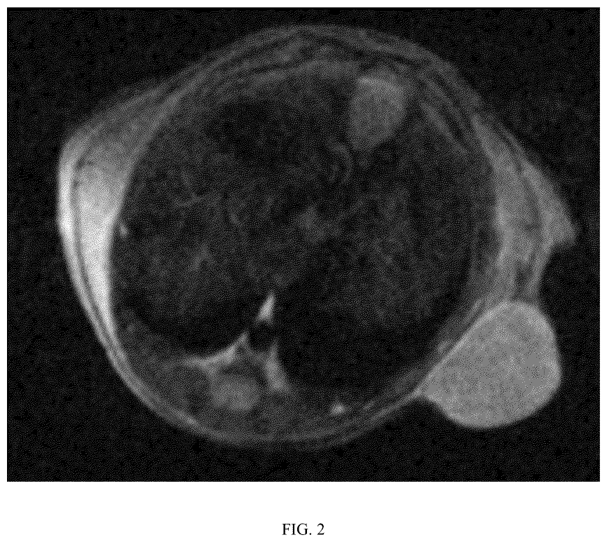

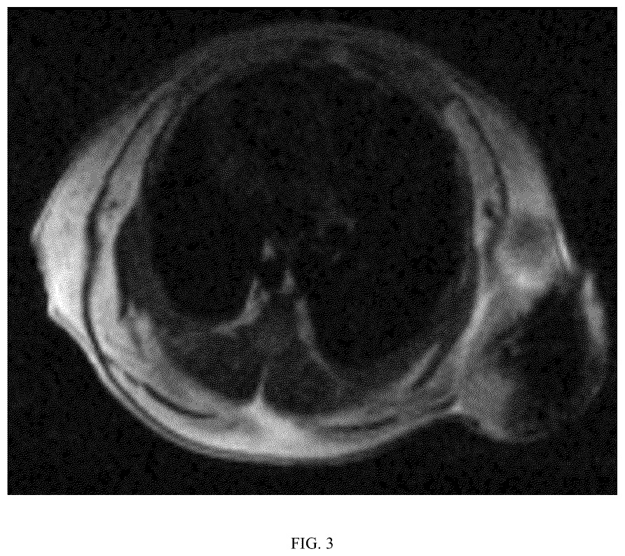

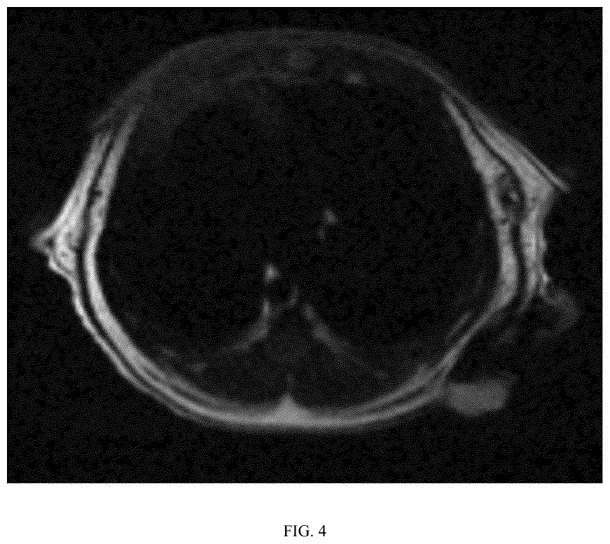

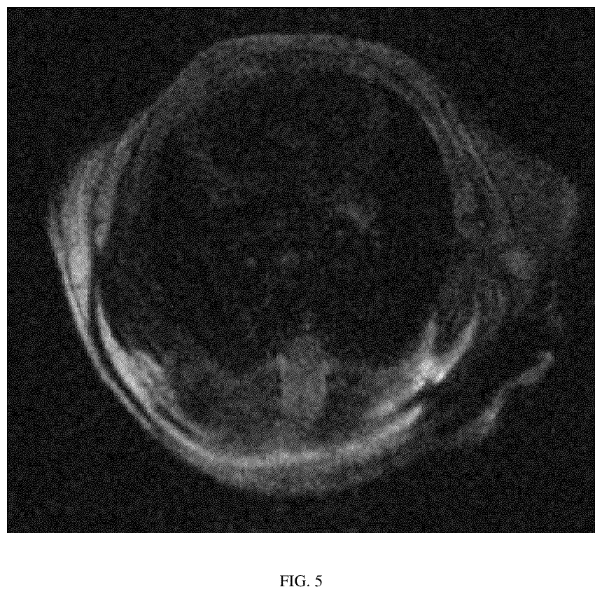

Test results: the mouse tumor is scanned with the 7T magnetic resonance scanning system for small animals, and scanning results are shown in FIG. 2, FIG. 3, FIG. 4 and FIG. 5. FIG. 2 shows an MRI image before intratumoral administration in the mice. FIG. 3 shows an MRI image on day 0 after intratumoral administration in the mice of the carbon nanoparticle suspension injection-Fe. The carbon nanoparticle suspension injection-Fe can be imaged in the magnetic resonance system after intratumoral administration, and the color is black. The carbon nanoparticle suspension injection-Fe is mainly distributed in the middle of tumor, and a small part on a periphery has no distribution of the carbon nanoparticle suspension injection-Fe. FIG. 4 shows an MRI image on day 3 after intratumoral administration in the mice of the carbon nanoparticle suspension injection-Fe, the tumor is slightly shrunk on day 3 after administration, there is a large amount of carbon nanoparticle suspension injection-Fe retained, which is mainly distributed in the middle, and a small part on the periphery has no distribution of the carbon nanoparticle suspension injection-Fe. FIG. 5 shows an MRI image on day 14 after intratumoral administration in the mice of the carbon nanoparticle suspension injection-Fe, the tumor is slightly shrunk on day 14 after administration, there is a large amount of carbon nanoparticle suspension injection-Fe retained, which is mainly distributed in the middle, and a small part on the periphery has no distribution of the carbon nanoparticle suspension injection-Fe.

The above test show that the carbon nanoparticle suspension injection-Fe is used in combination with magnetic resonance imaging, which can guide intratumoral administration of the carbon nanoparticle suspension injection-Fe, such that the carbon nanoparticle suspension injection-Fe is uniformly distributed in the tumor after administration, and the anticancer effect of the carbon nanoparticle suspension injection-Fe is improved. Moreover, the retention of the carbon nanoparticle suspension injection-Fe at different time after administration can also be observed through MRI, so as to know the amount and retention time of the carbon nanoparticle suspension injection-Fe in the tumor, and the retention time of the carbon nanoparticle suspension injection-Fe is long, which can effectively cover multiple mitotic cycles of cancer cells.

It should be understood that particular embodiments described above are merely used for illustratively describing or explaining the principle of the present disclosure, rather than constituting limitings to the present disclosure. Therefore, any modification, equivalent substitution, improvement, etc. made without departing from the spirit and the scope of the present disclosure should fall within the protection scope of the present disclosure. In addition, the claims appended of the present disclosure are intended to cover all changes and modifications that fall within the scope and boundaries of the appended claims, or equivalents of such ranges and boundaries.

1. Carbon nanoparticle suspension injection-Fe, comprising carbon nanoparticle suspension injection and ferrous sulfate, wherein a concentration of the carbon nanoparticle suspension injection is 20-100 mg/mL, and a concentration of ferrous ions in the carbon nanoparticle suspension injection-Fe is 0.5-60 mg/mL.

2. The carbon nanoparticle suspension injection-Fe according to claim 1, wherein the concentration of the carbon nanoparticle suspension injection is 50 mg/mL, and the concentration of the ferrous ions is 15-60 mg/mL.

3. The carbon nanoparticle suspension injection-Fe according to claim 1, wherein a particle size of carbon nanoparticles in the carbon nanoparticle suspension injection-Fe is 90-250 nm, and a pH value is 2.8-6.0.

4. The carbon nanoparticle suspension injection-Fe according to claim 1, wherein each 1000 mL of the carbon nanoparticle suspension injection comprises: 20-100 g of carbon nanoparticles, 17-30 g of poloxamer, 2-4 g of sodium citrate, 8-10 g of sodium chloride, 0.05-0.5 g of dimethicone, and the balance water for injection.

5. The carbon nanoparticle suspension injection-Fe according to claim 2, wherein each 1000 mL of the carbon nanoparticle suspension injection comprises: 50 g of carbon nanoparticles, 20 g of poloxamer, 0.2 g of dimethicone, 3 g of sodium citrate, 9 g of sodium chloride, and the balance water for injection.

6. A preparation method for carbon nanoparticle suspension injection-Fe, used for preparing the carbon nanoparticle suspension injection-Fe according to claim 1, comprising:
preparing carbon nanoparticles;
preparing each 20 mL of carbon nanoparticle suspension injection: selecting 400-2000 mg of carbon nanoparticles, 340-600 mg of poloxamer, 1-10 mg of dimethicone, 40-80 mg of sodium citrate, 160-200 mg of sodium chloride and the balance water for injection, performing homogenization for 5-10 minutes, and performing fine homogenization multiple times to prepare 20 mL of carbon nanoparticle suspension injection;
preparing each part of ferrous sulfate: dissolving 1490 mg of ferrous sulfate heptahydrate in 20 ml of water for injection, adjusting a pH value of the solution to 2.8, performing packaging, performing freeze-drying, backwashing nitrogen, and pressing lips, thereby obtaining ferrous sulfate for injection; and
sucking the carbon nanoparticle suspension injection, adding the carbon nanoparticle suspension injection into the ferrous sulfate for injection, and performing uniform mixing to obtain the carbon nanoparticle suspension injection-Fe.

7. The preparation method according to claim 6, wherein the preparing carbon nanoparticles comprises:
washing carbon nanoparticles with ethyl acetate, and then, performing filtering and drying;
washing the carbon nanoparticles with nitric acid, then, washing the carbon nanoparticles with water until a washing solution is neutral, and performing filtering and drying; and
washing the carbon nanoparticles with sodium hydroxide, then, washing the carbon nanoparticles with water until a washing solution is neutral, and performing filtering and drying.

8. The preparation method according to claim 6, wherein the sucking the carbon nanoparticle suspension injection, adding the carbon nanoparticle suspension injection into the ferrous sulfate for injection comprises:
in the case that the concentration of the ferrous ions is 60 mg/mL, taking 0.5 mL of carbon nanoparticle suspension injection, injecting same into a ferrous sulfate bottle under the condition of isolating air to be mixed, shaking same well, and shaking the mixture well after mixing to obtain the carbon nanoparticle suspension injection-Fe;
in the case that the concentration of the ferrous ions is 30 mg/mL, taking 1 mL of carbon nanoparticle suspension injection, injecting same into a ferrous sulfate bottle under the condition of isolating air to be mixed, shaking same well, and shaking the mixture well after mixing to obtain the carbon nanoparticle suspension injection-Fe;
in the case that the concentration of the ferrous ions is 15 mg/mL, taking 2 mL of carbon nanoparticle suspension injection, injecting same into a ferrous sulfate bottle under the condition of isolating air to be mixed, shaking same well, and shaking the mixture well after mixing to obtain the carbon nanoparticle suspension injection-Fe;
in the case that the concentration of the ferrous ions is 7.5 mg/mL, taking 4 mL of carbon nanoparticle suspension injection, injecting same into a ferrous sulfate bottle under the condition of isolating air to be mixed, shaking same well, and shaking the mixture well after mixing to obtain the carbon nanoparticle suspension injection-Fe; and
in the case that the concentration of the ferrous ions is 3.75 mg/mL, taking 8 mL of carbon nanoparticle suspension injection, injecting same into a ferrous sulfate bottle under the condition of isolating air to be mixed, shaking same well, and shaking the mixture well after mixing to obtain the carbon nanoparticle suspension injection-Fe.

9. An application of carbon nanoparticle suspension injection-Fe in magnetic resonance imaging development, in which the carbon nanoparticle suspension injection-Fe according to claim 1, wherein
the carbon nanoparticle suspension injection-Fe serves as a developing agent after being injected into a tumor to show a distribution area, a distribution amount and retention time of the carbon nanoparticle suspension injection-Fe on the surface of the tumor and/or in the tumor in a magnetic resonance image.

10. A use method of carbon nanoparticle suspension injection-Fe in magnetic resonance imaging development, employing the carbon nanoparticle suspension injection-Fe according to claim 1, and comprising:
determining a location and a size of a tumor for the first time through magnetic resonance imaging;
injecting the carbon nanoparticle suspension injection-Fe into the determined location of the tumor for the first time;
determining distribution of the carbon nanoparticle suspension injection-Fe in the tumor through magnetic resonance imaging by taking the carbon nanoparticle suspension injection-Fe as a developing agent; and
injecting the carbon nanoparticle suspension injection-Fe again at the tumor site where no or less the carbon nanoparticle suspension injection-Fe is distributed.

11. The use method according to claim 10, further comprising:
before each injection of the carbon nanoparticle suspension injection-Fe, determining the size and/or the location of the tumor and the distribution of the carbon nanoparticle suspension injection-Fe in the tumor through magnetic resonance imaging by taking the carbon nanoparticle suspension injection-Fe as the developing agent.

12. The use method according to claim 10, further comprising:
after each injection of the carbon nanoparticle suspension injection-Fe, determining the distribution of the carbon nanoparticle suspension injection-Fe in the tumor through magnetic resonance imaging by taking the carbon nanoparticle suspension injection-Fe as the developing agent.

13. The use method according to claim 10, wherein the distribution of the carbon nanoparticle suspension injection-Fe in the tumor comprises: a distribution area, a distribution amount and retention time of the carbon nanoparticle suspension injection-Fe on the surface of the tumor and/or in the tumor at different time points after injection.

14. The method of claim 6, wherein the concentration of the carbon nanoparticle suspension injection is 50 mg/mL, and the concentration of the ferrous ions is 15-60 mg/mL.

15. The method of claim 6, wherein a particle size of carbon nanoparticles in the carbon nanoparticle suspension injection-Fe is 90-250 nm, and a pH value is 2.8-6.0.

16. The method of claim 6, wherein each 1000 mL of the carbon nanoparticle suspension injection comprises: 20-100 g of carbon nanoparticles, 17-30 g of poloxamer, 2-4 g of sodium citrate, 8-10 g of sodium chloride, 0.05-0.5 g of dimethicone, and the balance water for injection.

17. The method of claim 14, wherein each 1000 mL of the carbon nanoparticle suspension injection comprises: 50 g of carbon nanoparticles, 20 g of poloxamer, 0.2 g of dimethicone, 3 g of sodium citrate, 9 g of sodium chloride, and the balance water for injection.

18. The application of claim 9, wherein the concentration of the carbon nanoparticle suspension injection is 50 mg/mL, and the concentration of the ferrous ions is 15-60 mg/mL.

19. The application of claim 9, wherein a particle size of carbon nanoparticles in the carbon nanoparticle suspension injection-Fe is 90-250 nm, and a pH value is 2.8-6.0.

20. The application of claim 9, wherein each 1000 mL of the carbon nanoparticle suspension injection comprises: 20-100 g of carbon nanoparticles, 17-30 g of poloxamer, 2-4 g of sodium citrate, 8-10 g of sodium chloride, 0.05-0.5 g of dimethicone, and the balance water for injection.
